(19) United States
(12) Patent Application Publication
(10) Pub. No.: US 2017/0209130 A1
(43) Pub. Date: Jul. 27, 2017

(54) DEVICE FOR TAKING SAMPLES OF TISSUE FOR CYTOLOGICAL/HISTOLOGICAL TESTS

(71) Applicant: Vincenzo CENNAMO, Bologna (IT)

(72) Inventor: Vincenzo CENNAMO, Bologna (IT)

(21) Appl. No.: 15/500,374

(22) PCT Filed: Jul. 22, 2015
(86) PCT No.: PCT/IB2015/055560

§ 371 Date: Jan. 30, 2017

(30) Foreign Application Priority Data
Jul. 31, 2014 (IT) BO2014A000437

(51) Int. Cl. A61B 10/02; A61B 10/00; A61B 10/04

(52) U.S. Cl. CPC A61B 10/0275; A61B 10/0233; A61B 10/02; A61B 10/04; A61B 10/00; A61B 2010/045; A61B 17/32053

(57) ABSTRACT

A device for taking samples of tissue for cytological/histological tests using an instrument, or endoscope, including a body provided with means for reversible association with the distal portion of the instrument; said body housing:

a needle for taking a tissue sample, movable between a proximal back, non-operating position, inside the body and a distal, operating position partly outside the body, for penetrating the tissue to be tested; said needle including a portion designed to form a seat for receiving said tissue sample. The device is also provided with first control means associated with said needle, being positioned and slidable inside said analysis instrument, and designed to allow the movement, in both directions, between said proximal position and said distal position.

### TECHNICAL FIELD

This invention relates to a device for taking samples of biological tissue for subsequent cytological/histological tests.

### BACKGROUND ART

This method is nowadays increasingly used in the routine practice of performing clinical tests on the human body.

For the analysis of internal organs, instruments commonly known as endoscopes are used for this purpose, such as, for example, may be seen in publication U.S. Pat. No. 5,861,002 or US-2005/0090842.

The samples are normally taken by using needles which can be inserted and housed inside the endoscope, and activated when the sample is taken.

To perform a needle biopsy echo-endoscopically, a flexible endoscope is used, fitted with an ultrasound probe which is inserted in the hollow organ to be examined.

In the case of an examination relating to the esophagus, stomach and/or duodenum and nearby organs (pancreas, bile ducts and lymph nodes), it is introduced through the oral cavity (as with the gastroscope), whilst in the case of an examination of the anal canal and/or rectum/sigmoid colon, it is introduced through the anus as far as necessary.

The ultrasound images obtained are displayed on a special screen which helps the operator to better maneuver the endoscopic instrument and position it appropriately in the immediate vicinity of the portion from which the sample is then taken.

During the examination it is therefore possible to carry out maneuvers such as the one indicated above, by using a needle which is placed in the operating channel of the instrument, to drain cysts or administer anesthetic at the solar plexus.

When a biopsy is being taken, to obtain correct tissue sampling, it may be necessary to operate the needle several times.

FIG. 1 of the accompanying drawings, and referring to a prior art solution, shows the distal portion (that is, the portion which is inserted inside the human body through the relative orifice) of the endoscope, the end part of which is also provided with elements for lighting and displaying 1 the part being examined.

Note the end of the instrument from which a needle 2 for FNA (Fine Needle Aspiration) projects from the operating channel and which is manoeuvred, at the appropriate moment, by the operator.

To perform the FNA, it is necessary to insert the needle in the endoscope channel until it protrudes from the distal portion of the endoscope.

The movements of the needle are then guided by the proximal end (not illustrated) of the handle which is equipped with a grip and controls associated with the various operating elements forming part of the endoscope.

In particular, the procedures which allow samples to be taken consist of:

- positioning the needle inside the instrument;

- inserting the needle in the lesion with a firm movement of the handle;

- modulating the excursion of the needle;

- aspiration of the material;

- removal of the needle by continuous movements;

- finally, withdrawing and removing the needle, and emptying of the needle to collect the sample.

The problem of the prior art is due to the limited possibility of altering the size and structure of the needle because of the need to pass through an endoscopic channel which, moreover, during the examination, adopts angled and curved positions not really suited for the transit of a needle-system, in one direction or another, inside it.

To have some idea of the shape and of functionalities of a needle-system, see, for example, the publication U.S. Pat. No. 5,199,441, whose main Figure is included as FIG. 2 of prior art in the accompanying drawings.

This solution clearly shows that the aspiration/sample taking needle is internally hollow so that this internal space can hold the tissue samples removed by insertion of the needle in the body part to be analysed.

To favour detachment and retention of the sample, the distal part of the outer surface of the needle is provided with holes passing through the thickness of the needle.

In addition, also to favour the retention of the tissue samples inside the needle, the cavity presents different thicknesses, thus creating a non-linear or undulating inner surface.

The drawbacks of the prior art consist therefore mainly in the fact that the needles used do not allow the removal of sufficient amounts of samples for in-depth analysis of the part under examination.

### DISCLOSURE OF THE INVENTION

The aim of this invention is to provide a device for taking samples of tissue for cytological/histological tests which overcomes the above-mentioned disadvantages of the prior art.

More specifically, the aim of this invention is to provide a device for taking samples of tissue for cytological/histological tests which can be associated with instruments of examination such as endoscopes, and is able to withdraw sufficient amounts of samples to allow completeness of the subsequent step of cytological/histological examination.

A further aim of this invention is propose a device for taking samples of tissue for cytological/histological tests capable of using, for its operation, the usual instruments and commands of traditional endoscopes, not requiring therefore particular designs, on one hand, or different instruments in the operating room, on the other.

### BRIEF DESCRIPTION OF DRAWINGS

This and other features of the invention will become more apparent from the following detailed description of a preferred, non-limiting example embodiment of it, with reference to the accompanying drawings, in which:

FIG. 1 illustrates an example of an instrument used for analyses, of the endoscope type, as a prior art solution;

FIG. 2 illustrates an example of a needle used for taking samples of tissue as a prior art solution;

FIGS. 3 and 4 are simplified views of the device according to this invention, connected to the end of an endoscope, as in practice, and in two different configurations;

FIGS. 5 to 8 show the device according to this invention in different configurations according to its use in situ;

FIG. 9 illustrates the device according to a simplified transversal cross-section;

FIG. 10 illustrates an example of the analysis instrument at its proximal end, with the control elements of the device according to the invention.

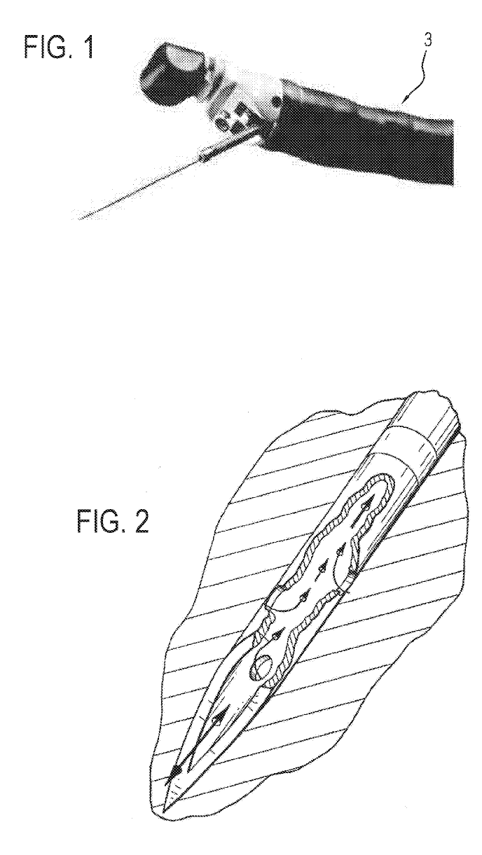

### DETAILED DESCRIPTION OF PREFERRED EMBODIMENTS OF THE INVENTION

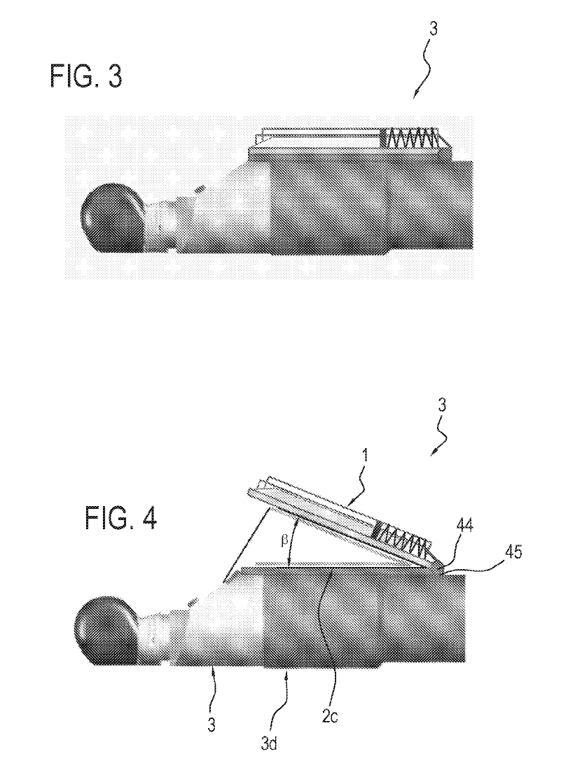

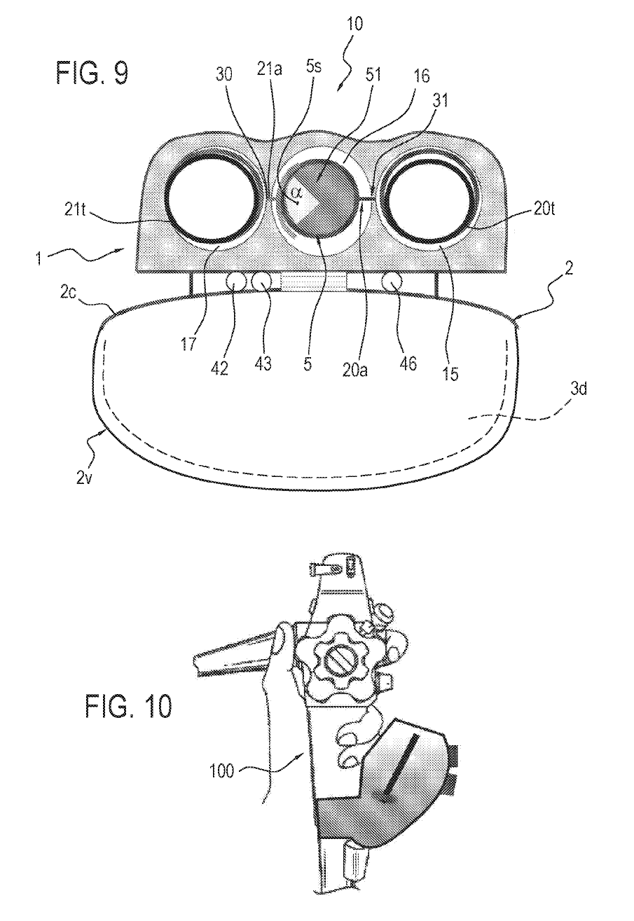

With reference to the accompanying drawings, and in particular to FIG. 9, the device according to this invention can be associated—as a complementary accessory—(see FIGS. 3 and 4) to a distal portion of an instrument for analysis of internal parts of the body of humans or animals, an instrument which is generically defined as an endoscope (3), whose proximal portion (see FIG. 10) ends with a handgrip (100) by means of which the surgeon/operator controls the various movements of the endoscope, as well as activating working parts such as lighting, cutting and retrieval of portions of tissue, etc. These functions are well known and are are not described in detail herein.

The device comprises a body (1) provided with means (2) for reversible association with the distal portion (3d) of the instrument (3).

These means are, in FIG. 9, made up of a cradle (2c) connected rigidly to or forming part of, the body (1), which may rest against the distal end (3d) of the endoscope and is fixed thereto by a strip (2v) for closing, preferably with elasticized material.

The body (1) houses a needle (5) for taking a tissue (6) sample, movable between a proximal back, non-operating position (5p), inside the body and a distal, operating position (5d) partly outside the body (1), for penetrating the tissue (6) to be tested.

The device is equipped with first control means (10) associated with said needle, being positioned and slidable inside said analysis instrument (controlled by a handle that can be attached to the handgrip positioned on the proximal part of the endoscope), and designed to allow the movement, in both directions, between said proximal position and said distal position.

In a preferred embodiment of the device according to the invention, the needle has a portion (5s) with reduced thickness to form a seat (7) for receiving said tissue sample.

In a preferred embodiment of the device according to the invention it comprises further movement means (better referred to below) designed to modify the orientation of the above-mentioned body (1).

The body also houses a first protection element (8) of the needle, surrounding the needle by at least a surface portion (8s) which is cylindrical and opposite the portion with reduced thickness (5s).

This first protection element is also positioned inside the body and is mobile, in both directions, parallel to a line of movement (9) of the needle, between a proximal, back, non-operating position (8p) and a distal, operating position (8d), in which the needle and first protection element are facing one another.

To realize this preferred combination of elements, the body (1) comprises, according to the solution illustrated in FIG. 9, three cavities (15, 16, 17), respectively side compartments (15, 17) and a central cavity (16).

The cavities are tubular and are connected to one another by relative longitudinal grooves (30, 31) cut in their lateral surface close to that of the adjacent cavity.

The groove (30) connects the side cavity (17) with the central cavity (16), whilst the groove (31) connects the remaining side cavity (15) with the central cavity (16).

The latter therefore has two longitudinal cavities opposite one another at 180°.

The central cavity (16) houses the needle (5) which is associated at the rear with a cylindrical support (51), said support forming the portion for needle coupling and sliding inside the cavity (16); see in particular FIG. 9.

The support (51) and the needle (5) comprise said portion (5s) with reduced thickness.

In FIG. 9 this portion (5s) with reduced thickness is defined as a cavity of the support (51) extending by approximately a right angle (α).

The side cavity (15) is internally provided with first actuator means (20) acting on the needle (5) to define its movement along the line of movement (9).

These first actuator means (20) are formed by a first cylindrical tubular body (20t), see FIG. 9, which acts as a slide inside the cavity (15) and which is securely connected to the support (51) of the needle (5) by a rib (20a) passing and slidable inside said groove (31).

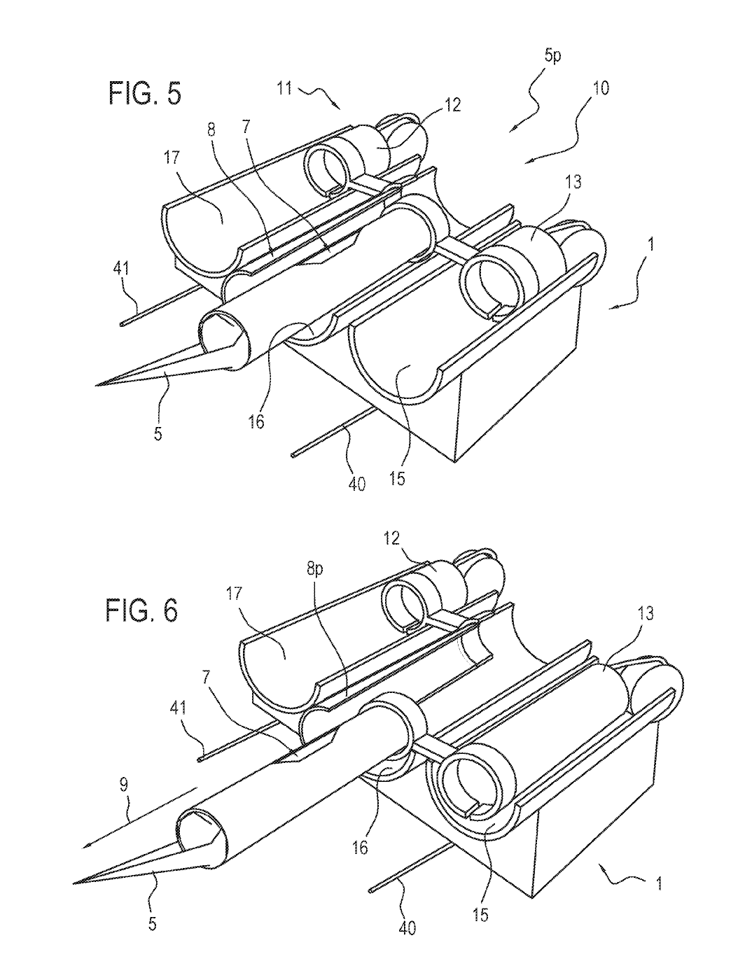

To complete the first actuator means (20), the device is provided with an elastic element, in the shape of a first spring (12), positioned at the back of the cavity (15) and acting on the back of said first cylindrical tubular body (20t); see also FIGS. 5 to 8.

The second side cavity (17) is internally provided with second actuator means (21) acting on the first protection element (8) to define its movement between the two positions, respectively proximal and distal.

These second actuator means (21) are formed by a second cylindrical tubular body (21t), see FIG. 9, which acts as a slide inside the cavity (17) and which is securely connected to the first protection element (8) of the needle (5) by a rib (21a) passing and slidable inside said groove (30).

To complete the second means actuators (21), the device is provided with an elastic element, in the shape of a second spring (13), positioned at the back of the cavity (17) and acting on the back of said second cylindrical tubular body (21t); see also FIGS. 5 to 8.

The reference numbers (40) and (41) denote first and second operating cables which slide inside the entire endoscope, usually using the existing ducts therein and which lead distally to said first spring (12) and said second spring (13) respectively, whilst proximally they are connected the handgrip (100) shown in FIG. 10.

More exactly, during the step of preparation of the endoscope instrument, the above-mentioned operating cables (40) and (41) are pulled taut in a distal-proximal direction, in such a way that they contribute to the compression of the first and second springs (12) and (13), this position of “arming” the device being made possible by blocking the proximal ends of the cables at the grip (100).

The aforesaid cables (40) and (41) pass through corresponding cable-holes denoted with (42) and (43), visible in FIG. 9.

The first actuator means (20), the first cylindrical tubular body (20t), the support (51) of the needle (5), the elastic element, in the shape of a first spring (12) and the first cable (40) define first control means labelled (10).

The second actuator means (21), the second cylindrical tubular body (21t), the first protection element (8) of the needle (5), the elastic element, in the shape of a second spring (13) and the second cable (41) define second control means labelled (11). We have seen that the device comprises the body (1) which is associated with the underlying cradle (2c).

In a preferred embodiment, shown in FIGS. 3 and 4, the body (1) oscillates with respect to the underlying cradle by means of a hinged coupling (44).

By means of this solution it is possible to move the body (1), and with it also the associated needle, between a lowered position of FIG. 3, in which the body (1) is parallel to the axis of the endoscope and a “ramp” position where the body (1), see FIG. 4, is raised at an angle, until reaching a value (β), in such a way as to favour the best position of the needle for the subsequent step of extracting and removing the tissue under examination.

In this case too, the movement of the ramp is carried out by means of a third cable (45), passing, like the previous ones, through a third hole (46), as shown in FIG. 9.

The functioning of the device according to the invention will now be briefly described.

During the preparation step, the body (1) is associated with, that is to say firmly attached to, the distal end of the endoscope, using the means (2v).

The first and second cables, (40) and (41), are pulled taut towards the handgrip (100) in such a way that the first and second springs (12) and (13) are compressed (loaded) with the needle (5) in a withdrawn position inside the relative cavity (16), see FIG. 5.

The first cylindrical tubular body (20t), (which is securely connected to the support (51) of the needle (5)) and the second cylindrical tubular body (21t), (which is securely connected to the first protection element (8) of the needle (5)) are also pulled taut and are located inside the respective cavities (15) and (17).

This static arrangement is due to the fact that the proximal end of the cables (40) and (41) is firmly fixed to the handgrip (100) of the endoscope by means of the usual locking techniques, for example with a choker and/or trigger.

At this point the endoscope is inserted in the relative orifice of the patient.

Once the correct position has been reached, the operator can activate the third cable (44) in such a way as to position the ramp at the right height, see FIG. 4.

By means of the trigger located on the handgrip, the operator can release the first cable (40), which leads to the protrusion, from the respective cavity (16), of the support (51) and needle (5), see FIG. 6.

The needle will penetrate the tissue to be examined, removing a certain amount of tissue which fills the seat (7) with reduced thickness (5s).

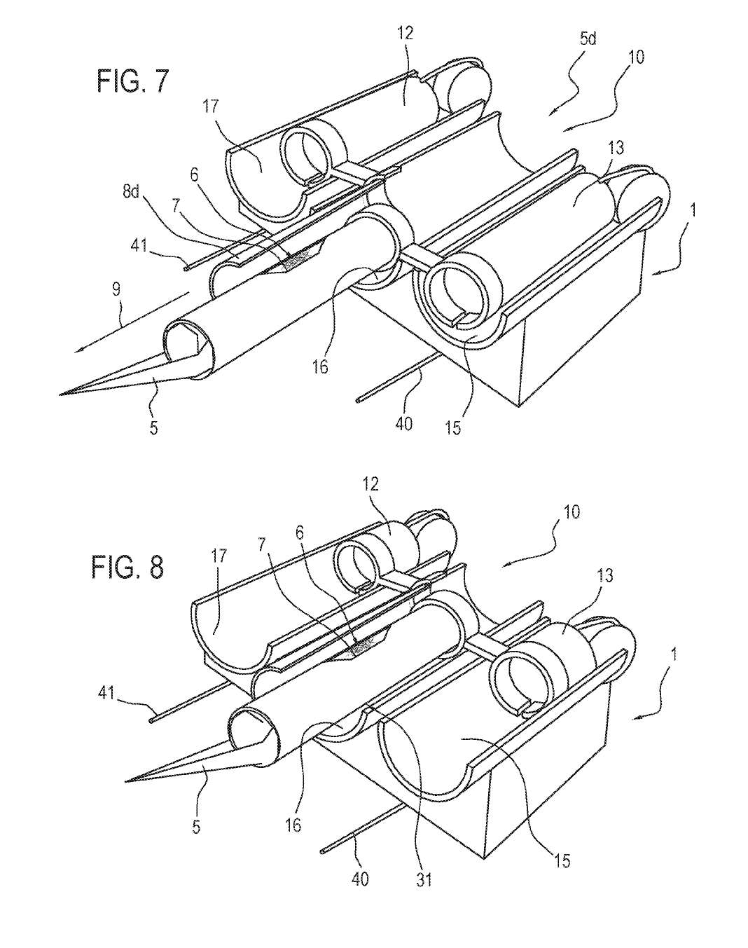

By means of the trigger on the handgrip, the operator then releases the second cable (41) with consequent expulsion from the respective cavity (17) of the protective element (8) which then covers and protects the needle (5) in such a way that the needle, but above all the zone of the seat (7), is partly enclosed, see FIG. 7.

The tissue sample taken from the patient is thus safely preserved.

The subsequent step consists of the operator applying traction on both of the cables (40) and (41) simultaneously, until all the movable parts are returned to their respective cavities: it should be noted that at this stage, the retraction of the needle-protective ensures preservation of the tissue sample taken, this being further protected inside the cavity (16), see FIG. 8.

At this point the entire endoscope can be withdrawn in order to remove the sample of tissue.

It is interesting to note that the device according to the invention can have different constructional types; the fundamental element consists of the independent body that can be associated with an analysis instrument. This embodiment allows the use of particular needles both in terms of size and shape, which cannot be used in traditional types of endoscope instruments. In a basic solution, the body might also have a single cavity inside which the needle is positioned. This may be the type indicated above (that is with a portion with reduced thickness) or the traditional type, that is with a hollow body, even if it has a large diameter.

The device according to this invention therefore achieves the preset aims since:
it can be associated with known types of examination instruments, such as endoscopes;
the needle can accommodate a greater amount of tissue sample than prior art instruments;
   and in the preferred embodiment illustrated in the Figures
it allows protection of this tissue sample inside a relative cavity until the endoscope is withdrawn at the end of the examination;
according to any embodiment, it allows orientation in situ of the position of the needle before the sample is taken.

1. A device for taking samples of tissue for cytological/histological tests using an instrument for analysing internal parts of the body of humans or animals, wherein it comprises a body provided with means for reversible association with the distal portion of the instrument; said body housing:
a needle for taking a tissue sample, movable between a proximal back, non-operating position, inside the body and a distal, operating position partly outside the body, for penetrating the tissue to be tested; said needle comprising a portion designed to form a seat for receiving said tissue sample;
first control means associated with said needle, being positioned and slidable inside said analysis instrument, and designed to allow the movement, in both directions, between said proximal position and said distal position.

2. The device according to claim 1, wherein the body comprises a first protection element of said needle, surrounding the outside of at least a cylindrical surface portion of the needle.

3. The device according to claim 1, wherein said needle comprises said portion designed to have a reduced thickness.

4. The device according to claim 2, wherein said cylindrical surface portion is opposite said portion with reduced thickness, said first protection element also being positioned inside the body and movable, in both directions, parallel to a line of movement of said needle, between a proximal, back, non-operating position and a distal, operating position in which the needle and said first protection element are facing one another.

5. The device for taking samples according to claim 2, comprising second control means associated with said protection element and positioned and slidable inside said analysis instrument, designed to allow the movement, in both directions, between said proximal position and said distal position.

6. The device for taking samples according to claim 3, wherein said portion with reduced thickness is a middle portion of the body of the needle.

7. The device for taking samples according to claim 1, wherein said body comprises first spring means positioned inside said body, acting on said needle for defining its movement from said proximal position to said distal position; and wherein first control means are associated with said needle, being positioned and slidable inside said analysis instrument, and designed to allow the movement between said distal position and said proximal position.

8. The device for taking samples according to claim 1, wherein said body comprises second spring means positioned inside said body, acting on said first protection element for defining its movement from said proximal position to said distal position and wherein second control means are associated with said first protection element, being positioned and slidable inside said analysis instrument, and designed to allow the movement between said distal position and said proximal position.

9. The device for taking samples according to claim 1, wherein said body comprises a set of three cavities, respectively lateral and central, which are connected to one another; said central cavity housing said needle; one of said lateral cavities being internally provided with first actuator means acting on said needle to define its movement along said line of movement; the second of said lateral cavities being internally provided with second actuator means acting on a first protection element for defining its movement between the two positions, respectively proximal and distal.

10. The device for taking samples according to claim 9, wherein the cavities are tubular and are connected to one another by related longitudinal grooves made at their lateral surface close to that of the adjacent cavity.

11. The device for taking samples according to claim 10, wherein the groove connects the lateral cavity with the central cavity, whilst the groove connects the remaining lateral cavity with the central cavity.

12. The device for taking samples according to claim 9, wherein said central cavity comprises two longitudinal cavities opposite one another at 180°.

13. The device for taking samples according to claim 9, wherein said central cavity houses the needle which is associated at the rear with a cylindrical support, said support forming the portion for needle coupling and sliding inside the cavity.

14. The device for taking samples according to claim 13, wherein the support, and with it the needle, comprise said portion with reduced thickness.

15. The device for taking samples according to claim 9, wherein said lateral cavity is internally provided with first actuator means acting on the needle to define its movement along the line of movement.

16. The device for taking samples according to claim 9, wherein said first actuator means are formed by a first cylindrical tubular body acting as a slide inside the cavity, said first tubular body being securely connected to the support of the needle by a rib passing and slidable inside said groove.

17. The device for taking samples according to claim 9, wherein it comprises an elastic element, in the shape of a first spring, positioned at the back of the cavity and acting on the back of said first cylindrical tubular body.

18. The device for taking samples according to claim 9, wherein said second lateral cavity is internally provided with second actuator means acting on the first protection element to define its movement between the two positions, respectively proximal and distal.

19. The device for taking samples according to claim 9, wherein said second actuator means are formed by a second cylindrical tubular body acting as a slide inside the cavity, said second tubular body being securely connected to the first protection element of the needle by a rib passing and slidable inside said groove.

20. The device for taking samples according to claim 9, wherein it comprises an elastic element, in the shape of a second spring, positioned at the back of the cavity and acting on the back of said second cylindrical tubular body.

21. The device for taking samples according to claim 9, comprising first and second operating cables and slidable inside the entire endoscope and, in a distal zone, respectively leading to said first spring and said second spring; in the proximal position the cables being connected to a handgrip of said instrument.

22. The device according to claim 1, wherein said body oscillates relative to a cradle below it, which is part of the means for reversible association, said oscillation being made possible by a hinged coupling.
